(19) United States
(12) Patent Application Publication
(10) Pub. No.: US 2022/0362401 A1
(43) Pub. Date: Nov. 17, 2022

(54) PARTICLES, METHOD FOR PRODUCING PARTICLES, DRUG, METHOD FOR PRODUCING DRUG, AND ANTI-CANCER AGENT

(71) Applicants: THE UNIVERSITY OF KITAKYUSHU, Kitakyushu-shi, Fukuoka (JP); Key Laboratory of Enzyme and Protein Technology, University of Science, Vietnam National University, Hanoi (VN)

(72) Inventors: Kazuo SAKURAI, Kitakyushu-shi, Fukuoka (JP); Shota FUJII, Kitakyushu-shi, Fukuoka (JP); Thi Hong Van Doan, Kitakyushu-shi, Fukuoka (JP); Thi Mai Phuong Nguyen, Hanoi (VN); Thi Van Anh Nguyen, Hanoi (VN); Thi Thu Huong Pham, Hanoi (VN)

(21) Appl. No.: 17/761,314

(22) PCT Filed: Aug. 25, 2020
(86) PCT No.: PCT/JP2020/032040

§ 371 Date: Mar. 17, 2022

(30) Foreign Application Priority Data
Sep. 20, 2019 (JP) 2019-172063

(51) Int. Cl. A61K 47/69; A61K 9/14; A61K 31/352; A61K 31/12; A61K 31/704

(52) U.S. Cl. CPC A61K 47/6951; A61K 9/14; A61K 31/352; A61K 31/12; A61K 31/704

(57) ABSTRACT

Particles using a polymer having a particle size suitable for a drug delivery system (DDS) or the like are provided. A drug and an anti-cancer agent using the particles are provided. Each of the particles comprises a polymer having a structure unit derived from a saccharide compound having a hydroxyl group and having an inclusion property and a structure unit derived from a monomer having a functional group to be reacted with a hydroxyl group, and has the average hydrodynamic radius (Rₕₐᵥ) of 5 to 100 nm. A method for producing the particles comprises a step of mixing a mixed composition comprising a saccharide compound, a monomer, a surfactant, and an alkaline aqueous solution having a pH of 12 or more. A drug compress a hydrophobic physiological active agent, such as α-mangostin contained in the particle, and an anti-cancer agent comprising the drug.

### TECHNICAL FIELD

The present invention relates to particles that can be used for a drug containing a hydrophobic physiological active agent and a method for producing the particles. The present invention relates to a drug using the particles and a method for producing the drug. The present invention also relates to an anti-cancer agent using these.

### BACKGROUND ART

It is known that some organic compounds called xanthones have an anti-cancer action. For example, α-mangostin and γ-mangostin, which are derivatives thereof, can be extracted from mangosteens, which are fruits native to Southeast Asia, as examples of xanthones that exhibit anti-cancer action.

Although the development of a new drug using high anti-cancer action of these mangostins has been required, it is difficult to deliver a drug only to target parts while suppressing the side effects thereof. A technique called the drug delivery system (DDS) in which a drug is delivered only to a target part using a polymer carrier attracts attention as a method for solving such a problem.

Meanwhile, a hyperbranched polymer comprising cyclodextrin and epichlorohydrin is known. The structure of this hyperbranched polymer is a spherical huge high polymer containing many branched structures. Since cyclodextrin contained in this polymer has hydrophobic cavities in its molecules, and has properties allowing to incorporate thereinto molecules with a suitable size and to carry various molecules, cyclodextrin attracts attention.

For example, Patent Literature 1 discloses a cyclodextrin crosslinked body obtained by crosslinking cyclodextrin with epihalohydrin under an alkaline condition. It is disclosed that this cyclodextrin crosslinked body is used as an environmental hormone removing agent or the like.

Patent Literature 2 discloses a composition, comprising:

an aqueous dispersion of particles with an average hydrodynamic size of 50 to 5000 nm (p), wherein (A) a polymer having an average content of at least four cyclodextrin units in structure and based on cyclodextrin units and (B) a polysaccharide high polymer having groups G in an average number of at least three per polysaccharide high polymer and comprising the groups G that can form an inclusion compound with cyclodextrin present in the structure of the polymer (A) are associated and contained in the particles, and the compounds (A) and (B) are water-soluble in isolation.

Non Patent Literature 1, Non Patent Literature 2, and the like disclose the examination of physical properties of a compound using cyclodextrin and epichlorohydrin, and the like. For example, in Non Patent Literature 1, the synthesis of an ionic cyclodextrin-based polymer and the examination of physical properties such as the incorporation capacity with an antimicrobial agent called triclosan have been mainly performed.

### CITATION LIST

### Patent Literature

Patent Literature 1

Japanese Patent Laid-Open No. 2003-226737
Patent Literature 2

Japanese Translation of PCT International Application Publication No. 2005-536587

### Non Patent Literature

Non Patent Literature 1

Carbohydrate Polymers 142 (2016) 149-157, Preparation and characterization of soluble branched ionic β-cyclodextrins and their inclusion complexes with triclosan.
Non Patent Literature 2

European Polymer Journal 46 (2010) 1915-1922, Water-soluble c-cyclodextrin polymers with high molecular weight and their complex forming properties.

### SUMMARY OF INVENTION

### Technical Problem

In the DDS technology, the particle sizes of polymeric carriers need to be a size that is not easily subjected to clearance (excretion) from the body, namely 10 nm or more in diameter (5 nm or more in radius), and it is required that target drugs be efficiently introduced into polymeric carriers.

As disclosed in Patent Literature 1 and Patent Literature 2, crosslinked bodies can be synthesized using cyclodextrin (hereinafter occasionally abbreviated as “CD”), epichlorohydrin (hereinafter occasionally abbreviated as “ECH”), and the like. Although methods for synthesis disclosed in these literatures proceed by stirring aqueous CD solutions and hydrophobic ECH vigorously, CD and ECH do not react efficiently due to heterogeneous reaction. Since the obtained products therefore vary in particle size, the products have very small sizes that are less than 10 nm in diameter (less than 5 nm in radius) and easily subjected to clearance from the body, or the products have large particle sizes, are captured, and cannot move in the living body.

When ECH is excessively added to enlarge the particle size, only ECH reacts intermolecularly, and an insoluble product is obtained. The reaction of CD and ECH needed to be precisely controlled to make the product water-soluble and enlarge the particle size to a certain size. The ring-opening reaction of ECH however had the property of proceeding at an increasing speed, and this control was very difficult.

Also in Patent Literature 2, the product is associated using a polysaccharide polymer, and the particle size tends to be enlarged. There is no example in which the introduction of a xanthone-based drug such as α-mangostin into this crosslinked body was attempted. It is believed that it is difficult to introduce a highly hydrophobic drug such as α-mangostin into a hydrophilic polymeric carrier.

An object of the present invention is to provide particles using a polymer having a particle size suitable for DDS or the like, and a method for producing the same.

### Solution to Problem

The present inventor has earnestly repeated investigation to solve the above-mentioned problem, consequently found that the following inventions are suitable for the above-mentioned object, and completed the present invention. That is, the present invention relates to the following inventions.

<A1> Particles, each comprising: a polymer having a structure unit derived from a saccharide compound having hydroxyl groups and having an inclusion property and a structure unit derived from a monomer having functional groups to be reacted with hydroxyl groups, wherein an average hydrodynamic radius (Rₕₐᵥ) is 5 to 100 nm.

<A2> The particles according to the <A1>, wherein the saccharide compound comprises a saccharide compound of any saccharide selected from the group consisting of cyclodextrin, β-1,3-glucan, and amylose.

<A3> The particles according to the <A1> or <A2>, wherein the monomer comprises a polyfunctional epoxy-based compound.

<A4> The particles according to any one of the <A1> to <A3>, wherein a value obtained by dividing standard deviation (σ) of particle size distribution by the average hydrodynamic radius (Rₕₐᵥ) (σ/Rₕₐᵥ) is 0.25 or less when the average hydrodynamic radius (Rₕₐᵥ) is defined as a median in the particle size distribution by normal distribution.

<A5> A drug, comprising: a hydrophobic physiological active agent contained in the particles according to any one of the <A1> to <A4>.

<A6> The drug according to the <A5>, wherein the hydrophobic physiological active agent comprises a hydrophobic physiological active agent dispersed in one or more dispersion media selected from the group consisting of dimethyl sulfoxide, tetrahydrofuran, N,N-dimethylformamide, methanol, ethanol, propanol, and butanol.

<A7> An anti-cancer agent, comprising: in the drug according to the <A5> or <A6>, a pharmaceutically acceptable carrier, and a hydrophobic physiological active agent of any selected from the group consisting of α-mangostin, curcumin, and doxorubicin.

<A8> The anti-cancer agent according to the <A7>, wherein the cancer is a solid cancer.

<B1> A method for producing particles,

comprising: a step of mixing a mixed composition comprising a saccharide compound having hydroxyl groups and having an inclusion property, a monomer having functional groups to be reacted with hydroxyl groups, a surfactant, and an alkaline aqueous solution having a pH of 12 or more,

wherein the particles each comprise a polymer formed in the mixed composition and having a structure unit derived from the saccharide compound and a structure unit derived from the monomer.

<B2> The method for producing particles according to the <B1>, wherein the surfactant comprises one or more surfactants selected from the group consisting of a quaternary ammonium salt-based surfactant, a sulfonate-based surfactant, and an oligo ethylene glycol-based surfactant.

<B3> The method for producing particles according to the <B2>, wherein the quaternary ammonium salt-based surfactant is hexadecyltrimethylammonium bromide, the sulfonate-based surfactant is sodium dodecyl sulfate, and the oligo ethylene glycol salt-based surfactant is Triton X-100.

<B4> The method for producing particles according to any one of the <B1> to <B3>, wherein a concentration of the surfactant in the mixed composition (mass of the surfactant/total mass of the mixed composition) is 4 times or less a critical micelle concentration of the surfactant in neutral water.

<B5> A method for producing a drug, wherein a first composition comprising the particles obtained by the production method according to any one of the <B1> to <B4> and a second composition comprising a hydrophobic physiological active agent are mixed to incorporate the hydrophobic physiological active agent into the particles.

<B6> The method for producing a drug according to the <B5>, wherein the second composition comprises one or more dispersion media selected from the group consisting of dimethyl sulfoxide, tetrahydrofuran, N,N-dimethylformamide, methanol, ethanol, propanol, and butanol as a dispersion medium.

### Advantageous Effects of Invention

Particles of the present invention have a particle size suitable for a DDS or the like. According to a method for producing the particles of the present invention, such particles can be obtained efficiently.

### BRIEF DESCRIPTION OF DRAWINGS

FIG. 1 is a flow chart for describing a process for producing particles in the Examples.

FIG. 2 is a graph showing the particle size distributions of the particles depending on whether SDS is present or not according to Example 1.

FIG. 3 is a graph showing the particle size distributions of particles according to Production Example 1.

FIG. 4 is a graph showing the particle size distribution of particles according to Production Example

FIG. 5 is graphs showing the results of a test in which α-mangostin is incorporated according to Production Example 3.

FIG. 6 is a graph showing the particle size distributions of particles according to Production Example 5.

FIG. 7 is a graph showing the results of incorporation tests of drugs according to Production Example 7.

FIG. 8 is a graph showing the evaluation results of the cell viabilities of colorectal cancer cells in vitro according to Example 2.

FIG. 9 is a graph showing the results obtained by evaluating the effect of suppressing colorectal cancer cell proliferation in mice according to Example 3.

### DESCRIPTION OF EMBODIMENTS

Although embodiments of the present invention will be described in detail hereinafter, the description of components described hereinafter is examples (representative examples) of the embodiments of the present invention. The present invention is not limited to the following contents as long as the gist thereof is not changed. When the expression “to” is used herein, the expression is used as an expression wherein the numerical values on the right and left sides thereof are inclusive.

[Particles of the Present Invention]

Particles of the present invention each contain a polymer having a structure unit derived from a saccharide compound having hydroxyl groups and having the inclusion property and a structure unit derived from a monomer having functional groups to be reacted with hydroxyl groups, and has an average hydrodynamic radius (Rₕₐᵥ) of 5 to 100 nm. Since the particles of the present invention can contain a hydrophobic compound or the like, and have such a particle size, the particles have a size suitable for a DDS or the like.

[Method for Producing Particles of the Present Invention]

A method for producing particles of the present invention is a method for producing particles, comprising: a step of mixing a mixed composition comprising a saccharide compound having hydroxyl groups and having an inclusion property, a monomer having functional groups to be reacted with hydroxyl groups, a surfactant, and an alkaline aqueous solution having a pH of 12 or more, wherein the particles each comprise a polymer formed in the mixed composition and having a structure unit derived from the saccharide compound and a structure unit derived from the monomer. Particles the hydrodynamic radius of which is controlled to around 5 to 100 nm can be produced efficiently by producing the particles by such a production method.

The method for producing particles of the present invention is a suitable method for producing particles of the present invention, and the configurations corresponding to respective configurations can be mutually used in the present application.

[Saccharide Compound]

In the method for producing particles of the present invention, the saccharide compound having hydroxyl groups and having an inclusion property is used as one of the raw materials. The particles of the present invention each have a structure unit derived from the saccharide compound having hydroxyl groups and having the inclusion property. The polymer is formed using the monomer to be used for producing the particles of the present invention with such a saccharide compound to form large particles.

These particles can contain the hydrophobic substance or the like.

The saccharide compound contains a sugar and a sugar derivative. A monosaccharide, an oligosaccharide, a polysaccharide, or the like can be used as the sugar. For example, a cyclic oligosaccharide or the like in which glucose molecules are connected by α-1,4-glycosidic bonds or α-1,6-glycosidic bonds, and a glycosidic bond is formed between the reducing end and the final end is preferably used. There is cyclodextrin (CD) as a representative example of such a cyclic oligosaccharide. The sugar is not limited to these, and, for example, cyclic amylose or the like described in Bulletin of Applied Glycoscience, vol. 1, No. 1, 39-46 (2011) and the citations thereof can also be used.

Sugar derivatives contained in the saccharide compound are compounds in which these sugars are partially modified or substituted. As the derivative, a sugar or a polysaccharide in which some hydroxyl groups are methylated, aminated or sulfonated can be used. The derivative is specifically carboxycellulose, hydroxyethylcellulose, hydroxypropylmethylcellulose, or the like. A similar modification of a polysaccharide other than cellulose can also be utilized. The saccharide compound has hydroxyl groups, and becomes a crosslinked body in which these hydroxyl groups are crosslinked through the monomer. The hydroxyl groups that the molecular structure of the saccharide compound has are preferably two or more. The hydroxyl groups may be three or more.

The saccharide compound to be used for the particles or the like of the present invention is preferably a saccharide compound of any saccharide selected from the group consisting of CD, β-1,3-glucan, and amylose. CD is a cyclic oligosaccharide in which a plurality of glucose molecules is bound with glycosidic bonds and that has a cyclic structure. CD includes hydrophobic molecules.

Any CD of α-cyclodextrin (cyclohexaamylose) (α-CD), in which six glucose molecules are bound, β-cyclodextrin (cycloheptaamylose) (β-CD), in which seven glucose molecules are bound, and γ-cyclodextrin (cyclooctaamylose) (γ-CD), in which eight glucose molecules are bound, or the like may be used.

β-1,3-glucan is a polysaccharide in which D-glucose molecules are connected by glycosidic bonds, and constitutes laminaran or the like, contained in seaweed or mushrooms. Amylose is a polysaccharide in which α-glucose molecules are connected by glycoside bonds that are α1→4 bonds, and is contained in starch or the like.

These sugars may be used alone or in combination optionally. These sugars are suitable for controlling the particle size of the polymer to be used for the particles of the present invention, and are also excellent in the property of incorporating a hydrophobic compound or the like. These sugars have low toxicity, and are easily used as a part of the raw materials ingested by humans, animals, or the like.

[Monomer]

The monomer having functional groups to be reacted with hydroxyl groups is used for the method for producing particles of the present invention. The monomer is preferably a polyfunctional epoxy-based compound. Here, “having two or more functional groups” is called “polyfunctional”. It is preferable to use especially a monomer such as epihalohydrin, ethylene glycol diglycidyl ether, or butanediol diglycidyl ether having functional groups to be reacted with hydroxyl groups at a plurality of sites as one of the raw materials. The number of the functional groups that are reacted with hydroxyl groups and that the monomer has is preferably two or more, may be three or more, or four or more. The particles of the present invention each have a structure unit derived from the monomer having the functional groups to be reacted with hydroxyl groups. This monomer reacts with hydroxyl groups of the above-mentioned saccharide compound to form a crosslinked body.

As such a monomer, epihalohydrin is preferably used. It is preferable to use especially epichlorohydrin (ECH) and/or epibromohydrin, and it is further preferable to use ECH. Epihalohydrin is suitable for forming a polymer having crosslinked structure by causing the reaction in which hydroxyl groups of the saccharide compound to be used for the present invention are crosslinked.

[Polymer]

The particles of the present invention each have the polymer having the structure unit derived from the saccharide compound having hydroxyl groups and having the inclusion property and the structure unit derived from the monomer having functional groups to be reacted with hydroxyl groups. For example, this polymer has the structure unit derived from the saccharide compound and the structure unit derived from the monomer. For example, when CD is used as the saccharide compound, and ECH is used as the monomer, these are reacted to obtain a crosslinked polymer branched very greatly (hyperbranched polymer). Formula 1 is a hypothetical example of the chemical structural formula of the polymer constituted by such CD and ECH. In Formula 1, n is the number of glucose molecules contained in cyclodextrin (CD). When n is 6, the CD corresponds to α-CD. When n is 7, the CD corresponds to β-CD. When n is 8, the CD corresponds to γ-CD.

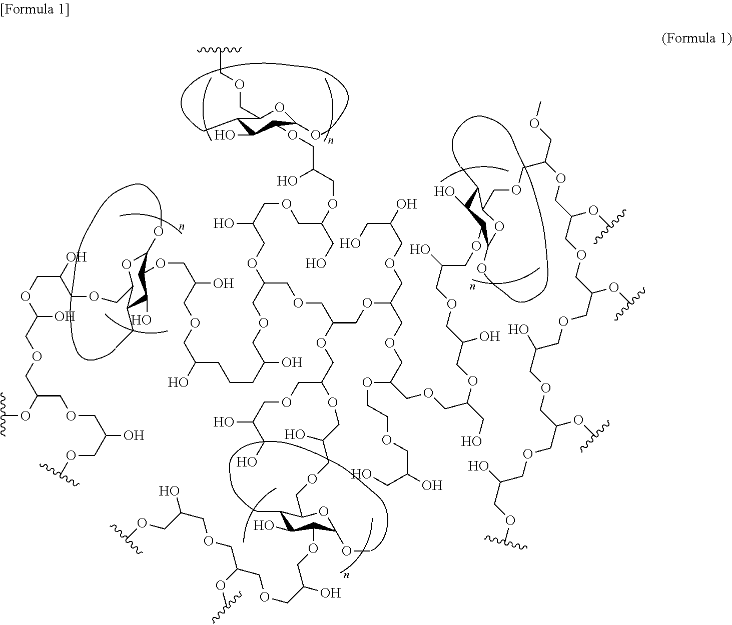

The polymer each substantially comprising the structure unit derived from the saccharide compound having the hydroxyl groups and having the inclusion property and the structure unit derived from the monomer having the functional groups to be reacted with hydroxyl groups can be used for the particles of the present invention. The management and control with the components from which the structure units are derived are easily performed without needing other components for enlarging the particle size by forming the polymer having such a constitution. The phrase “substantially comprising these structure units” here means that the above-mentioned structure unit derived from the saccharide compound and the above-mentioned structure unit derived from the monomer account for a very high rate in the polymer structure. As long as the object of the particles of the present invention is not deteriorated, impurities that can be contained in the production process or the like or substances that the structure units have at terminals, side chains, or the like in a very small amount are however contained. For example, when the rate of the above-mentioned structure unit derived from the saccharide compound and the above-mentioned structure unit derived from the monomer is 80% by mass or more, or 90% by mass or more at the time of polymer structure analysis, this may be defined as an index of whether the polymer substantially comprises these structure units. This rate may be 95% by mass or more, or 98% by mass or more.

[Particle Shape and the Like]

The particles of the present invention contain such a polymer. The polymer particles produced by controlling the particle size using the method for producing particles of the present invention allow to selectively obtain particles having a hydrodynamic radius of 5 to 100 nm. When the hydrodynamic radius is less than 5 nm, the size is a size that is easily removed by various physiological phenomena such as urine and sweat and the movement thereof between cells due to the metabolism or the like in the living body. When the hydrodynamic radius is adjusted to 5 nm or more, these removals hardly occur, and these particles can reach the predetermined tissue and cells in the living body to subject an active component to sustained release by incorporating the active component such as a drug into the particles. When the hydrodynamic radius exceeds 100 nm, the particles may be captured in the living body to be unmovable, or the application to the formulation into powder, tablets, or the like may be difficult.

This average particle size and its distribution can be found by particle size distribution analysis using dynamic light scattering. A standard method described in “Hikari Sanran-Hou no Kiso to Ouyou (Fundamentals and Application of Light Scattering)” (KODANSHA LTD.) can be used with respect to the measuring method. Specifically, particles dispersed in a solution are irradiated with laser light, and the scattered light thereof is measured with a photon detector. Since particles are constantly moving the positions thereof due to Brownian motion, the intensity distribution due to the interference of scattered light is also constantly fluctuating. This appearance of Brownian motion is observed as the fluctuation of the scattered light intensity. An autocorrelation function is usually found by the photon correlation method, and the diffusion coefficient, which indicates the Brownian motion velocity, and additionally the particle size and the particle size distribution can be found using the cumulant method, the histogram analysis method, and the CONTIN analysis. The present invention is based on the CONTIN analysis. A program attached to a particle size measuring device, or the like can be used for the CONTIN analysis.

In the particles of the present invention, the average hydrodynamic radius (Rₕₐᵥ) (hereinafter abbreviated as the “average particle size”) of the main product, which gives the main peak in the particle size distribution of the particles, is 5 to 100 nm. The lower limit of the average particle size of the particles of the present invention is preferably 10 nm or more, more preferably 12 nm or more, further preferably 15 nm or more, and particularly preferably 20 nm or more. The upper limit of the average particle size of the particles of the present invention is preferably 80 nm or less, more preferably 50 nm or less, further preferably less than 50 nm, and particularly preferably 30 nm or less.

The particle size distribution is preferably a single-peaked type, which has one peak.

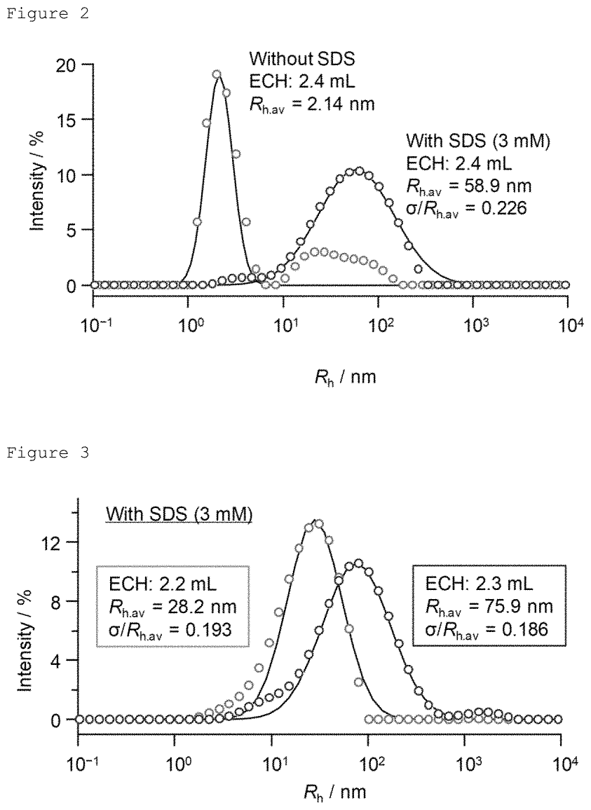

When the average particle size (Rₕₐᵥ) is defined as a median in the particle size distribution in the normal distribution, a value obtained by dividing the standard deviation (σ) by the average particle size (σ/Rₕₐᵥ) is preferably 0.25 or less. In the method for the determination thereof, the obtained scattering intensity (intensity) and hydrodynamic radius Rh are plotted with the vertical axis showing the intensity and the horizontal axis showing Rh on a logarithmic scale, a Gaussian distribution is assumed in this graph, and the standard deviation σ is found by peak fitting. Then, σ/Rₕₐᵥ is calculated. σ/Rₕₐᵥ of FIGS. 2 to 4 and 6 is thus calculated values. The value obtained by dividing the standard deviation (σ) by the average particle size (σ/Rₕₐᵥ) is more preferably 0.23 or less, and further preferably 0.20 or less.

[Drug]

A drug in which a hydrophobic physiological active agent is incorporated into the particles of the present invention can be obtained. If hydrophobic physiological active agents are hydrophobic compounds that can be dissolved in an organic solvent that can be mixed with water at any ratio, all the hydrophobic physiological active agents can be used for this drug. Examples of such hydrophobic physiological active agents include hydrophobic physiological active agents described in Chem. Pharm. Bull. 66, 493-505 (2018).

[Hydrophobic Physiological Active Agent]

The particles of the present invention can contain the hydrophobic physiological active agent. The hydrophobic physiological active agent is a hydrophobic compound having physiological activity. Such a compound is hardly dispersed in water. Even when the compound is expected to exhibit various physiological activities for drugs, functional foods, and the like, such a compound may not have a suitable mixing method as a drug component, or may be difficultly controlled by the DDS. According to the present invention, incorporation of such a hydrophobic physiological active agent allows to control a tissue in the body to which the active agent is released, time to exhibit a sustained-release property, and the like. Examples of the hydrophobic physiological active agent include xanthone derivatives, polyphenols, glucosides and flavonols. Examples of these include physiological active substance such as xanthone derivatives such as α-mangostin, polyphenols such as curcumin, stilbene derivative polyphenols such as resveratrol, and 2,3,5,4-tetrahydroxy-diphenylethylene-2-O-glucoside (THSG); and flavonols such as dihydromyricetin, and astilbin.

[α-Mangostin]

Examples of the hydrophobic physiological active agent include α-mangostin. α-mangostin is one of xanthones contained in the husks of mangosteen, which is an evergreen tall tree of Garcinia. Formula 2 shows the chemical structural formula of α-mangostin. α-mangostin produces a cancer suppression effect.

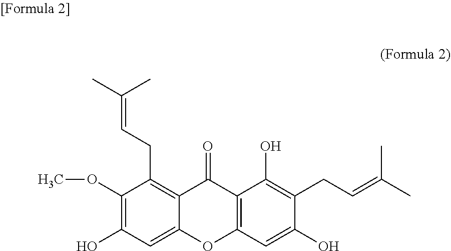 [SMILES: COc1c(O)cc2oc3cc(O)c(CC=C(C)C)c(O)c3c(=O)c2c1CC=C(C)C]

The particles of the present invention may be made to contain a physiological active agent or the like as they are and used as a drug, or may be used as particles for drugs for incorporating a physiological active agent or the like into the particles to use the particle. A particle size equivalent to the particles of the present invention can be maintained even after the physiological active agent is incorporated although the particle size depends on the physiological active agent or its inclusion conditions. The particles of the present invention collected after filtration, drying, centrifugation, and the like may be used as they are. The particles of the present invention may be stored or used in a state in which the particles are easily used in any aspect such as a dispersion or a suspension in which these particles is dispersed or suspended in liquid, powder or tablets molded by mixing these particles with various vehicles or the like, or capsules.

[Anti-Cancer Agent]

An anti-cancer agent of the present invention can contain the hydrophobic physiological active agent of the drug according to the present invention that is any selected from the group consisting of α-mangostin, curcumin, and doxorubicin as an active ingredient. The anti-cancer agent can further contain a pharmaceutically acceptable carrier optionally. The drug can be used for producing the anti-cancer agent. The drug can be the drug to be used in treatment using the anti-cancer agent. The therapeutic method can be a therapeutic method for cancer comprising administering the therapeutically effective amount of the drug to a patient needing treatment.

Since the active ingredient of the anti-cancer agent such as α-mangostin has a low molecular weight, the active ingredient is promptly removed from the body by metabolisms such as excretion from the kidney to urine. The use of the particles of the present invention having a size of a hydrodynamic radius of 5 nm or more makes it difficult for the particles to be removed from the body by renal excretion or the like. The active ingredient can therefore remain in blood or the like for a long period of time. Since tumor tissues increase in blood vessel permeability as compared with normal tissues, particles having a hydrodynamic radius of around 5 to 100 nm easily leak out of the blood vessels. Since the lymphatic systems of tumor tissues is underdeveloped, particles hardly disappear, and tend to accumulate. Such a phenomenon is known as an enhanced permeability and retention (EPR) effect. The particles of the present invention can avoid renal excretion, and have a size suitable for this EPR effect. A remarkable anti-tumor effect (anti-cancer effect) is obtained.

The anti-cancer agent of the present invention can be used for humans, mammals, animals, and the like. When the anti-cancer agent of the present invention is used for humans, the daily dosage of α-mangostin, curcumin, or doxorubicin, which is a hydrophobic physiological active agent, and is an active ingredient, can be the following. The daily dosage can be 0.5 mg/(kg·day) to 20 mg/(kg·day) per a body weight of 1 kg. That is, this dosage can be 30 mg/day to 1200 mg/day in terms of an adult having a body weight of 60 kg. The lower limit of this dosage can be preferably 1.0 mg/(kg·day) or more, or 1.5 mg/(kg·day) or more per a body weight of 1 kg as a daily dosage. The upper limit of this dosage can be preferably 15.0 mg/(kg·day) or less, or 10.0 mg/(kg·day) or less per a body weight of 1 kg as a daily dosage.

The anti-cancer agent of the present invention can be administered by a use such as injection, endermic administration, or oral administration depending on the target cancer. Especially since the anti-cancer agent can remain for enough time to produce effect, the anti-cancer agent is preferably administered by intravenous injection. The anti-cancer agent of the present invention can be suitably prepared as a solid preparation or a liquid preparation containing a pharmaceutically acceptable carrier as used commonly depending on these administration methods.

As the dosage form of the anticancer agent, specific examples of the solid preparation include powder drugs, granules, tablets, capsules, and troches. As a liquid preparation, liquid agents for internal use, liquid agents for external use, suspensions, emulsions, syrups, injections, infusions, and the like are illustrated, and these dosage forms and other dosage forms are suitably selected depending on the object.

In a solid preparation, adjuvants such as a vehicle, a binder, a disintegrator, a lubricant, a flavor, and a stabilizer may be used with the main ingredient. The ratio of the main ingredient to the adjuvants is suitably selected depending on the object. Examples of the vehicle in the solid preparation include lactose, D-mannitol, and starch. Examples of the binder include crystalline cellulose, white sugar, D-mannitol, dextrin, and hydroxypropylcellulose. Example of the disintegrator include starch, carboxymethylcellulose, and carboxymethylcellulose calcium. Preparation additives such as an antiseptic, an antioxidant, a coloring agent, and a sweetener may be used if needed.

When the anti-cancer agent is used as the liquid preparation, a solvent that disperses the active ingredient or the drug, and is safe for the living body is selected. Suitable examples of the solvent include water for injection, ethanol, and propylene glycol.

The liquid preparation may contain auxiliary components such as a solubilizing agent, a suspending agent, an isotonizing agent, a buffer, and an antioxidant with the main ingredient. Examples of the solubilizing agent include ethanol, polyethylene glycol, propylene glycol, benzyl benzoate, sodium carbonate, and sodium citrate. Examples of the suspending agent include sodium lauryl sulfate, laurylaminopropionic acid, lecithin, benzalkonium chloride, benzethonium chloride, glyceryl monostearate, polyvinyl alcohol, polyvinyl pyrrolidone, and hydroxymethylcellulose. Examples of the buffer include buffer solutions of phosphates, acetates, carbonates, and the like. Examples of the antioxidant include sulfites and ascorbates.

The anti-cancer agent of the present invention can target various cancers. Especially since the anti-cancer agent remains in blood to produce an effect on a tissue or an internal organ for a long period of time, the anti-cancer agent preferably targets solid cancers. If solid cancers are illustrated, examples of the solid cancers include cancers produced from epithelial cells (epithelial tumors) and sarcomata, produced from nonepithelial cells. Representative examples of the cancers include lung cancer, breast cancer, stomach cancer, colorectal cancer, uterine cancer, ovarian cancer, laryngeal cancer, pharyngeal cancer, lingual cancer, osteosarcoma, chondrosarcoma, rhabdomyosarcoma, leiomyosarcoma, fibrosarcoma, liposarcoma, and angiosarcoma.

[Method for Producing Particles of the Present Invention]

The method for producing particles of the present invention is a method for producing particles, comprising: a step of mixing a mixed composition comprising a saccharide compound having hydroxyl groups and having an inclusion property, a monomer having functional groups to be reacted with hydroxyl groups, a surfactant, and an alkaline aqueous solution having a pH of 12 or more, wherein the particles each comprise a polymer formed in the mixed composition and having a structure unit derived from the saccharide compound and a structure unit derived from the monomer.

[Mixed Composition]

The method for producing particles of the present invention comprises the step of mixing the mixed composition comprising the saccharide compound having hydroxyl groups and having the inclusion property, the monomer having the functional groups to be reacted with hydroxyl groups, the surfactant, and the alkaline aqueous solution having a pH of 12 or more. This mixed composition can be reacted under the predetermined conditions to obtain the particles that are the polymer having the structure unit derived from the saccharide compound and the structure unit derived from the monomer.

The mixed composition can be prepared by mixing such as the addition of the saccharide compound and the surfactant to be used for the particles of the present invention to the alkaline aqueous solution having a pH of 12 or more and subsequent addition of the monomer to be used for the particles of the present invention. This order or combination may be optionally changed. The above-mentioned saccharide compounds or monomers are suitably used as these saccharide compounds or monomers.

[Surfactant]

Various surfactants such as anionic surfactants, cationic surfactants, and neutral surfactants can be used alone, or can be optionally mixed and used as the surfactant. Although CD and ECH can be reacted in the alkaline aqueous solution to obtain very small particulates, this size is a very small size that is a hydrodynamic radius of 0.5 nm to 2.5 nm. Particles stably having a hydrodynamic radius of 5 nm or more were not obtained in the past. The present inventors have found that the size can be controlled even to an average particle size of 5 nm or more using the surfactant in combination here. It is believed that this is because surfactants contain hydrophilic moieties, dissolved in water, and hydrophobic moieties, dissolved in oil, the functional appearance as a phase transfer catalyst that moves ECH or the like to the aqueous phase can be expected, and the monomer such as ECH, and CD contact easily, and a crosslinked body grows easily.

The mixed composition preferably contains one or more surfactants selected from the group consisting of a quaternary ammonium salt-based surfactant, a sulfonate-based surfactant, and an oligo ethylene glycol-based surfactant. These surfactants may be used alone or in combination of two or more. It is believed that when the method for producing particles of the present invention is performed, the critical micelle concentration of the surfactant is preferably higher. The surfactants as mentioned above have physical properties such as critical micelle concentrations suitable for the present invention.

Even though any of the surfactants are used, the particle size of the obtained hyperbranched polymer is as large as 5 nm or more in average particle size, which cannot be achieved in conventional synthesis methods. The particle size is controllable by the molar ratio between ECH and CD to be added.

[Quaternary Ammonium Salt-Based Surfactant]

As the surfactant, a quaternary ammonium salt-based surfactant can be used appropriately. As the quaternary ammonium salt-based surfactant, for example, hexadecyltrimethylammonium bromide (hereinafter referred to as “CTAB”) can be used appropriately.

Tetradecyltrimethylammonium bromide and octadecyltrimethylammonium bromide, which are different in alkyl chain length, hexadecyltrimethylammonium chloride, which is different in the counter ion, and the like can also be used.

[Sulfonate-Based Surfactant]

As the surfactant, the sulfonate-based surfactant can be used appropriately. As the sulfonate-based surfactant, for example, sodium dodecyl sulfate (hereinafter referred to as “SDS”) can be used appropriately.

Sodium decyl sulfate and sodium tetradecyl sulfate, which are different in alkyl chain length, potassium dodecyl sulfate, which is different in the counter ions, and the like can also be used.

[Oligo Ethylene Glycol Salt-Based Surfactant]

As the surfactant, the oligo ethylene glycol salt-based surfactant can be used. As the oligo ethylene glycol-based surfactant, for example, Triton X-100 (hereinafter referred to as “TX100”) can be used appropriately.

Triton X-114, which is different in polyoxyethylene chain length, TWEEN 20, which is a similar nonionic surfactant, and the like can also be used.

[Surfactant Concentration]

The surfactant concentration can be a very important factor for adjusting the particle size. Although the concentration varies also depending on the surfactant type, the concentration is preferably 4 times or less the critical micelle concentration (hereinafter referred to as “CMC”) in neutral water. Particularly preferably, the particle size can be freely controlled using the surfactant at the CMC or less. When the surfactant is not added, ECH separates from the solvent, and the reaction proceeding is heterogeneous. It is however found that even though ECH is added, the mixture becomes a translucent solution, and ECH is dispersed homogeneously at a moderate surfactant concentration. Although a surfactant is dissolved as molecules or ionized ions at a certain concentration or less, those associate to form micelles, and exhibits properties as a colloidal solution at a specific concentration or more. This boundary concentration is called the CMC of a surfactant.

Values described in literatures or values measured as CMCs with respect to conditions to be used if needed are used as CMCs by surfactants to be used. For example, the CMC of hexadecyltrimethylammonium bromide is around 1.0 mM. The CMC of SDS is around 8.0 mM. The CMC of TX100 is around 0.23 mM.

[Alkaline Aqueous Solution]

The mixed composition contains the alkaline aqueous solution having a pH of 12 or more. When the saccharide compound and the monomer are reacted in the presence of the alkaline aqueous solution, the saccharide compound and the monomer exhibit crosslinking reaction to form the polymer. The pH of the alkaline aqueous solution is preferably 13 or more, and more preferably 13.5 or more. For example, water in which hydroxides of alkaline metals of sodium hydroxide (NaOH), lithium hydroxide (LiOH), potassium hydroxide (KOH), and the like; and tetraalkylammonium are mixed can be used. Sodium hydroxide (NaOH) can be appropriately used especially from the viewpoint of availability and the like. The concentration of the hydroxide in the alkaline aqueous solution can be 1% by mass or more or a concentration as high as 5% by mass or more, 10% by mass or more, or 20% by mass or more.

[Mixed Composition]

The concentration of the saccharide compound in the mixed composition (the mass of the saccharide compound/the total mass of the mixed composition) is around 5 to 20% by mass. When the concentration of the saccharide compound is too low, the particles may not grow to a sufficient size. When the concentration of the saccharide compound is too high, the particles may become too large; the ratio thereof to other components is a ratio at which crosslinking hardly occurs, and the particles may not grow; or precipitate may be produced without being dissolved.

The concentration of the monomer in the mixed composition (the mass of the monomer/the total mass of the mixed composition) is around 40 to 90% by mass. When the concentration of the monomer is too low, the particles may not grow to a sufficient size.

When the concentration of the monomer is too high, the particles may be too large; the ratio thereof to other components is a ratio at which crosslinking hardly occurs, and the particles may not grow; or the monomer cannot be fully dispersed in and compatible with the mixed composition, and may separate on the surface not to be reacted.

Although the concentration of the surfactant in the mixed composition (the mass of the surfactant/the total mass of the mixed composition) varies also depending on the surfactant type, the concentration is preferably 4 times or less the critical micelle concentration (CMC) of the surfactant in neutral water, and the particle size can be freely controlled using the surfactant more preferably at twice or less the CMC, further preferably at the CMC or less. When the lower limit is set with respect to the concentration, the lower limit is preferably 1/20 or more of the CMC, or may be more preferably 1/15 or more of the CMC, 1/10 or more of the CMC, or ⅕ or more of the CMC.

When the concentration of the surfactant is too low, the monomer cannot be dispersed in and compatible with the mixed composition, and the particles may not grow to a sufficient size. When the concentration of the surfactant is too high, the surfactant alone may form micelles, the effect of the surfactant may not be obtained, and the particles may not grow.

The weight ratio of the saccharide compound to the monomer in the mixed composition (saccharide compound:monomer) is suitably set in the range of around 10:90 to 20:80. The weight ratio is preferable 13:87 to 17:83. If either of the saccharide compound and the monomer is present in a large amount, the crosslinking reaction hardly occurs, and the particles may not grow to a sufficient size.

To enlarge the particle size of the particles, the enlargement is feasible, for example, by increasing the weight ratio of the monomer to be added or adjusting the reaction temperature. The most important point in the present invention is the amount of the surfactant to be added. As a standard, the use of the surfactant at a concentration such as around 4 times or less the CMC, or the CMC or less based on the CMC measured in a neutral salt-free aqueous solution is particularly effective. The number of monomers per one emulsion particle dispersed by the surfactant decreases at a concentration still higher than the CMC, the mixed composition may not be able to become large particles after the polymerization.

Meanwhile, it is believed that since the monomer functions as cores, and makes the surfactant gather by itself at the CMC or less, the number of monomers per one emulsion particle increases, and the particles after the polymerization are consequently stabilized, and become large. As presumed from this reaction mechanism, an optimal surfactant concentration, and the concentrations of the monomer and the saccharide compound, and the composition ratio between the monomer and the saccharide compound that are suitable therefor vary depending on the target particle size. The concentrations are however preferably around 4 times or less the CMC based on the CMC usually measured in a neutral salt-free aqueous solution.

The alkaline water solution concentration in the mixed composition (the mass of the alkaline aqueous solution/the total mass of mixed composition) is preferably 35% by mass or less. The alkaline aqueous solution is contained as a reaction site, and is the remainder except the saccharide compound, the monomer, the surfactant, and other additive components. When the alkaline aqueous solution concentration is too low, hydroxyl groups of the saccharide compound and the monomer is not fully activated, the reaction is not promoted, and the particles may not therefore be generated. When the alkaline aqueous solution concentration is too high, hydroxyl groups of the reactant is so activated that the control of the reaction is difficult, and the target particles may not be obtained.

[Reaction Conditions]

The mixed composition can be prepared and then reacted under the predetermined conditions to obtain the particles of the present invention. It is preferable to suitably react the mixed composition with stirring using a stirrer, a stirring blade, or the like during the reaction.

This reaction can also be performed at around the normal temperature, or may be performed under a heating condition or a cooling condition optionally. For example, the mixed composition can be reacted at 0° C. to 100° C., at which the operation is easy even at normal pressure using the alkaline aqueous solution. This temperature is preferably 10° C. or more, preferably 20° C. or more, and more preferably 25° C. or more. When the temperature is too low, the reaction may be slow, or the surfactant or the like may not function fully. The upper limit of the temperature may be in the range such as 80° C. or less, 60° C. or less, or 50° C. or less, in which the mixed composition is not excessively heated.

The reaction time can be suitably adjusted depending on the combination of the saccharide compound and the monomer for forming the particles, the particle size, reaction temperature, or the like. The reaction time in the method for producing particles of the present invention is preferably be performed with relatively long time spent for the growth of the particles. The reaction can be performed for 1 hour or more, 2 hours or more, 3 hours or more, or 6 hours or more. Since it is believed that the reaction time is finished in a certain range, and the reaction reaches the state in which the growth of the particles, or the like does not occur, the upper limit such as 100 hours or less and 50 hours or less, or 30 hours or less may be set as the upper limit of the reaction time.

After the reaction, a neutralizing liquid (acidic aqueous solution or the like) for neutralizing the reaction liquid is mixed into the mixed composition in which the particles are formed, and the reaction is stopped completely. The mixture can then be mixed with a nonpolar solvent such as acetone and reprecipitated to isolate only the particles. The molecular weight and particle size of the obtained particles are suitably evaluated by static and dynamic light scattering measurement.

[Incorporation of Hydrophobic Physiological Active Agent]

An incorporation step of incorporating the hydrophobic physiological active agent into the particles of the present invention can be performed by mixing a first composition containing the particles of the present invention and a second composition containing a hydrophobic physiological active agent to produce a drug in which the hydrophobic physiological active agent is contained in the particles of the present invention.

[First Composition]

The first composition contains the particles of the present invention. This first composition may be the particles of the present invention alone or a mixture thereof with other dispersion media or the like, and a concept including these is defined as the first composition in the present application. The particles of the present invention are dispersed in water. When a dispersion medium is used, it is therefore preferable to use a polar solvent such as water or a lower alcohol having around 1 to 4 carbon atoms.

[Second Composition]

The second composition contains the hydrophobic physiological active agent. This second composition may be the hydrophobic physiological active agent alone or a mixture thereof with other dispersion media, and a concept including these is defined as the second composition in the present application. It is preferable to use a dispersion medium such as liquid, or the like for mixing the first composition and the second composition, and at least the second composition preferably contains the dispersion medium.

An organic solvent that can be mixed with water at any ratio is preferably used for the dispersion medium of the second composition. The dispersion medium of the second composition preferably contains one or more dispersion media selected from the group consisting of dimethyl sulfoxide (hereinafter referred to as “DMSO”), tetrahydrofuran, N,N-dimethylformamide, methanol, ethanol, propanol, and butanol. These dispersion media exhibit amphipathicity, and are suitable for dispersing the physiological active agent having hydrophobicity. These dispersion media are also suitable for mixing and dispersing the first composition, containing the particles of the present invention.

Thus, the first composition and the second composition are mixed, and the particles of the present invention and the hydrophobic physiological active agent are contacted in the mixed dispersion medium to incorporate the hydrophobic physiological active agent into the particles of the present invention. This mixing for incorporation can be performed at around 10 to 50° C., or may be performed at around 20 to 40° C. The reaction time can be around 0.5 hours to 24 hours, or may be around 1 hour to 12 hours.

Formula 3 shows a reaction scheme for synthesizing the particles as a hyperbranched polymer using CD and ECH and a scheme for introducing α-mangostin into the particle. ECH and CD can be reacted in the alkaline aqueous solution containing the surfactant and using a 33% by mass NaOH solution at 30° C. for 6 hours to obtain the polymer. This polymer is particles having the predetermined particle size. α-mangostin can be mixed with these particles in the presence of a solvent such as DMSO, and α-mangostin and these particles are contacted with stirring at room temperature for 2 hours to obtain a mixture in which α-mangostin is contained in the particles having the predetermined particle size.

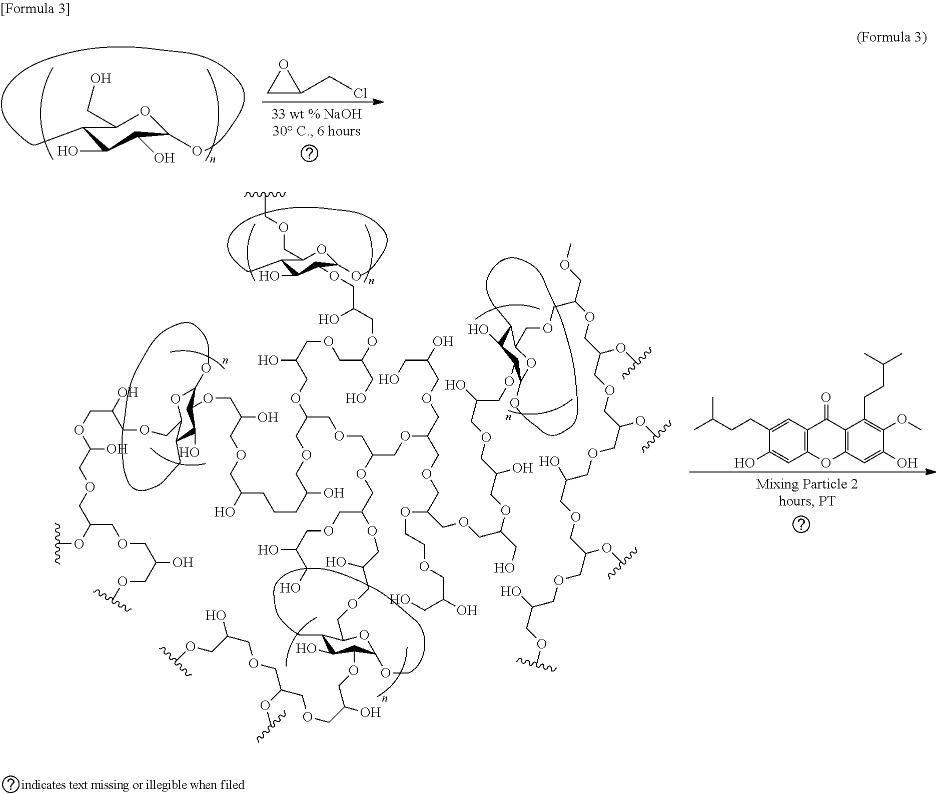

The reaction will be described in more detail by exemplifying the reaction according to Formula 3. A preparation method in which an α-mangostin/DMSO solution, in which α-mangostin is dissolved in DMSO as a water-soluble organic solvent that exhibits high affinity for the polymerized particles, is added is adopted as a method of introducing (incorporating) α-mangostin into particles.

CD is insoluble in DMSO. Even though an α-mangostin/DMSO solution is added to an aqueous CD solution, the incorporation rate remains low. If CD is prepared into the particles of the present invention, even the particles having the CD structure unit however improves in the dispersibility in DMSO remarkably, and it can therefore be expected that α-mangostin is efficiently introduced into CD in the polymer through DMSO.

This mixture is performed, for example, by adding a small amount of a 10 mg/mL solution of α-mangostin in DMSO to an aqueous solution of the particles of the present invention, using CD. Since α-mangostin that is not incorporated into the particles of the present invention precipitates at this time, the α-mangostin is removed by centrifugation.

It can be confirmed by FFF-UV/RI measurement to which flow field fractionation and an ultraviolet and visible spectrophotometer/differential refractometer are connected that α-mangostin is contained in the particles of the present invention. The rate of α-mangostin loaded in the particles of the present invention can be calculated from the UV absorbance.

When α-mangostin is introduced (incorporated) into the particles of the present invention by the method according to the present invention, a high loading rate can be achieved. Even though this is compared with polymeric micelles commonly used as other polymeric carriers, this is around 10 times higher.

### EXAMPLES

The present invention will be described by the Examples in further detail hereinafter. As long as the gist thereof is not changed, the present invention is not however limited to the following Examples.

[Evaluation Items]

### [Particle Size (Average Particle Size and Particle Size Distribution)]

The particle size was measured using a “DelsaMax PRO” manufactured by Beckman Coulter, Inc. The autocorrelation function obtained by dynamic light scattering measurement was subjected to CONTIN analysis using the attached software to specify the particle size. The main measurement conditions and analysis conditions at the time of measuring the particle size of particles are as follows: sample concentration: 5.0 mg/mL, solvent: water, the number of measurements: 5 times, temperature: 25° C., scattering angle: 172°, and wavelength: 532 nm.

Hereinafter, the average hydrodynamic radius (Rₕₐᵥ) is defined as the “average particle size”. When the average hydrodynamic radius (Rₕₐᵥ) is defined as a median in the particle size distribution by the normal distribution, a value obtained by dividing the standard deviation (σ) by the average hydrodynamic radius (Rₕₐᵥ) (σ/Rₕₐᵥ) is defined as one of indices that indicate the degree of distribution.

[Incorporation Property]

The incorporation of the hydrophobic physiological active agent into the particles was evaluated using “FFF-UV/RI measurement to which an asymmetric flow field fractionator (FFF) and an ultraviolet and visible spectrophotometer (UV)/differential refractometer (RI) are connected”, manufactured by Wyatt Technology Corporation.

[Loading Rate]

The rate of the hydrophobic physiological active agent loaded in the particles was measured using a “V-600”, manufactured by JASCO Corporation.

### [Reagent and the Like]

### Cyclodextrin (CD): Tokyo Chemical Industry Co., Ltd.

Epichlorohydrin compounds (ECH): Tokyo Chemical Industry Co., Ltd.
Sodium hydroxide (NaOH): FUJIFILM Wako Pure Chemical Corporation
Hexadecyltrimethylammonium bromide: Tokyo Chemical Industry Co., Ltd.
Sodium dodecyl sulfate (SDS): FUJIFILM Wako Pure Chemical Corporation

### Triton X-100 (TX100): NACALAI TESQUE, INC.

α-mangostin: Tokyo Chemical Industry Co., Ltd.
Water: ultrapure water
Dimethyl sulfoxide (DMSO): FUJIFILM Wako Pure Chemical Corporation

### [Example 1] Production of Particles

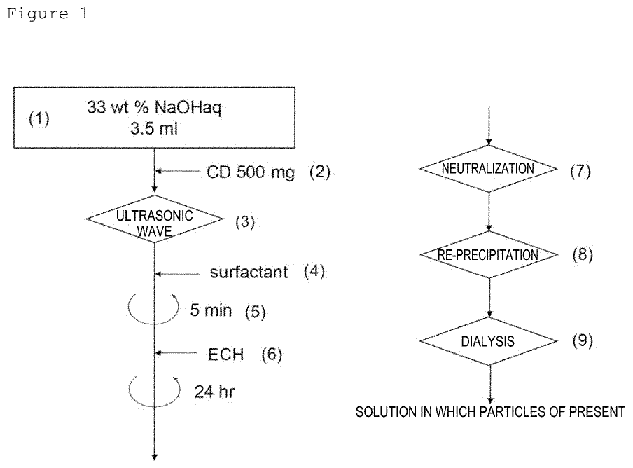

A method for producing particles was performed according to the flow chart shown in FIG. 1. The experiment procedure is as follows.

(1): An aqueous 33% by mass (wt %) NaOH solution (water: 3.2 g, NaOH: 1.6 g) is provided.
(2): Then, 500 mg of β-CD is dissolved in (1).
(3): Next, β-CD is dispersed by ultrasonic waves.
(4): Subsequently, 100 μL of an SDS solution (98.5 mM (“mmol/L” is abbreviated as merely “mM”) stock solution) (surfactant) is added to the solution of (3). Since 3.2 g of water has already been present, the concentration of SDS is 3 mM. This is defined as X (first variation parameter) hereinafter.
(5): The mixture is stirred at room temperature for 5 minutes. The mixture is stirred with a magnetic stirrer.
(6): Then, 2.4 mL of ECH is added while the solution is stirred. This value is defined as Y (second variation parameter) hereinafter. The mixture is stirred at room temperature for 24 hours.
(7): After the stirring for 24 hours, the reaction solution is neutralized.
(8): Reprecipitation is performed (good solvent: water, poor solvent: acetone).
(9): The reprecipitated substance was collected and dialyzed. A Spectra/Por® Standard Regenerated Cellulose (RC) Membrane having a cutoff molecular weight of 3.5 kD was used in the dialysis. The dialyzed substance was freeze-dried for 3 to 4 days after the dialysis to obtain the particles of the present invention.

FIG. 2 is the particle size distribution of the obtained particles. If the variance σ/Rₕₐᵥ was determined from the peak, the variance σ/Rₕₐᵥ was 0.226, and the distribution was single-peaked. The average particle size determined from the local maximum of the peak was 59 nm. Hereinafter, a case where the variance is 0.25 or less, and the particles are monodispersed, and the average particle size is in the range of 10 nm to 100 nm is indicated as “excellent”.

As shown in FIG. 2, particles having an average particle size of around 2 nm is generated without SDS, and large particles are further present at around 20 nm. Thus, the distribution has usually a profile with multiple peaks, which has a plurality of peaks. This is because the epoxy-based monomer has a plurality of reactive sites, the reaction increases continuously, and the control is therefore difficult. In the system to which the surfactant is added and that has been found in the present invention, the surfactant meanwhile functions as a phase transfer catalyst, controls the reaction, and gives uniform and single-peaked distribution. Although examples will be shown below, the average particle size obtained when SDS is added so that the concentration is 3 mM is around 60 nm is very large here.

As seen above, the surfactant can be added to produce the polymer that uses CD and in which the particle size is controlled.

### Production Example 1

The compositions of SDS and ECH were changed in completely the same way except that X and Y were changed in Example 1. Table 1 shows the combinations at that time. The distribution was measured by the same method as in Example 1. The case where either condition of a condition that the distribution is single-peaked and a condition that the variance is 0.25 or less is not satisfied, and the main peak is however in the range of 10 to 100 nm is indicated as “good”. The case where the distribution was two-peaked, and the variance exceeded 0.25 was indicated with “poor”, and the case where the average particle diameter was less than 10 nm, or exceeded 100 nm was also indicated as “poor”. Table 1 shows the results.

As shown in Table 1, ideal particles are obtained in the combination of specific X and Y. FIG. 3 shows the figure of the particle size distributions of the obtained particles. When SDS was 0 mM, the average particle diameter was less than 10 nm, and was evaluated as “poor”. The distribution might exhibit a profile with multiple peaks, or the variance might increase depending on the balance between the variation parameter X and the variation parameter Y.

As shown also in FIG. 3, in the case of 2.2 mL of ECH, the average particle is around 28 nm, and in the case of 2.3 mL of ECH, particles having an average particle size of around 76 nm are obtained.

TABLE 1
Variation parameter Y

| | | ECH | | | | | |
|---|---|---|---|---|---|---|---|
| | SDS | 2.1 ml | 2.2 ml | 2.3 ml | 2.4 ml | 2.5 ml | |
| Variation | 0 mM | Poor | Poor | Poor | Poor | Poor | Excellent → single peak |
| parameter | 2.0 mM | Good | Good | Good | Excellent | Poor | and variance of 0.25 or |
| X | (CMC × 0.25) | | | | | | less |
| | 3.0 mM | Good | Excellent | Excellent | Excellent | Poor | |
| | (CMC × 0.38) | | | | | | |
| | 4.0 mM | Good | Good | Good | Excellent | Poor | Good → two peaks or |
| | (CMC × 0.50) | | | | | | variance of 0.25 or more |
| | [symbol] mM | Good | Good | Excellent | Excellent | Good | |
| | (CMC × 0.75) | | | | | | |
| | 8.0 mM | Good | Good | Excellent | Excellent | Excellent | Poor → multiple peaks |
| | (CMC) | | | | | | |
| | 40 mM | Poor | Poor | Poor | Poor | Poor | |
| | (CMC × [symbol] ) | | | | | | |

[symbol]

indicates data missing or illegible when filed

### Production Example 2

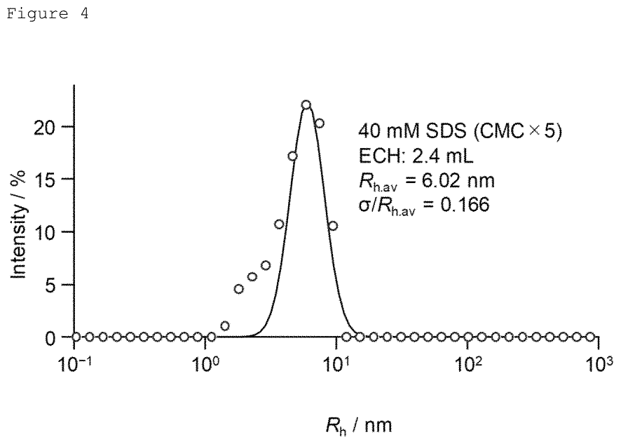

The SDS concentration was 5 times the CMC in Example 1. FIG. 4 shows the results obtained by evaluating the particle size by the same method as in Example. The average particle size of particles obtained under this condition was around 6 nm, and the particles were particles having a small average particle size.

### Production Example 3

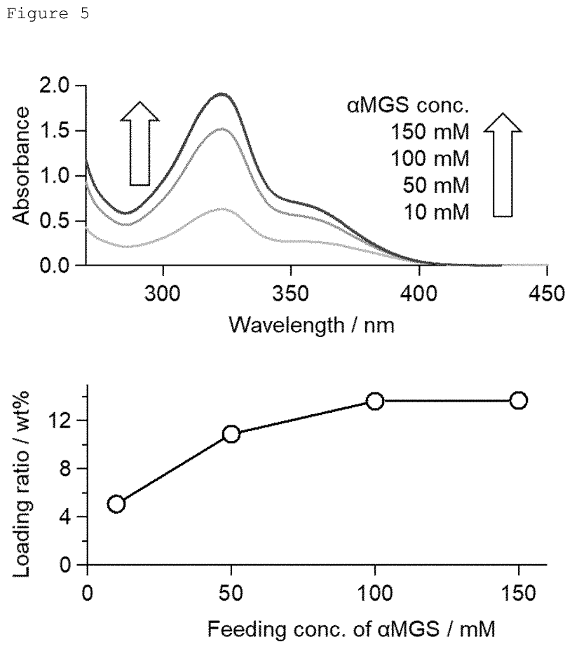

α-mangostin was incorporated into the particles obtained in Example 1. Then, 50 μL of a solution of α-mangostin in DMSO (100 mM: this value is a third variation parameter. In FIG. 5, this value is indicated as “Feeding cocn. of αMGS”) was added to an aqueous particulate solution (10 mg/mL, 450 μL), and the mixture was then left to stand at room temperature for 2 hours. Then, α-mangostin that precipitated without being contained in the particulates was removed by centrifugation. DMSO in the aqueous solution was removed by performing ultrafiltration operation 3 times. The obtained aqueous solution was subjected to FFF-UV/RI measurement and UV-vis measurement to confirm the incorporation of α-mangostin into the particulates and determine the loading rate. The incorporation was performed by the same method with the concentration of the solution of α-mangostin in DMSO changed from 10 mM to 150 mM. FIG. 5 shows the results.

The upper figure of FIG. 5 is data of the UV absorption. There was no absorption at all before the incorporation of α-mangostin, and α-mangostin has UV absorption at this wavelength (323 nm). The incorporation rate per unit weight can therefore be calculated from the absorbance. The lower figure of FIG. 5 shows the calculated results. A loading rate of around 14 wt % is achieved. This is a very high value as compared with the following Production Example 4.

### Production Example 4

First, 450 μL of 10 mg/mL β-CD was provided, and 50 μL of a solution of α-mangostin in DMSO (concentration: 100 mM) was added. The precipitated α-mangostin was removed by centrifugation. When the loading rate was measured by the same method as in Production Example 3 after the removal of DMSO by dialysis, the loading rate was 2 to 3%.

### Production Example 5

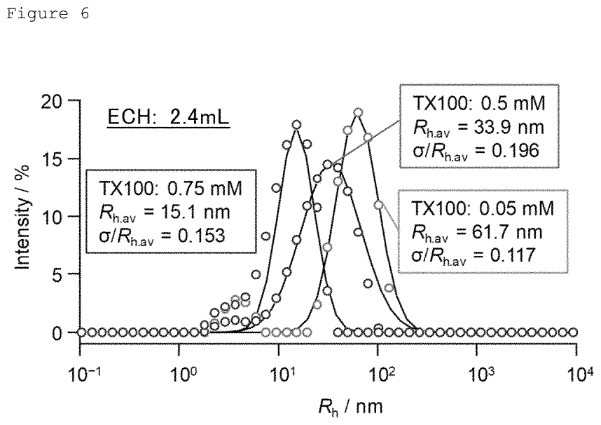

Particles were produced in the same way as in Example 1 except that the values of the first variation parameter were changed, and the surfactant type was changed into TX100. Table 2 shows the evaluation results. FIG. 6 shows measurement examples of particle size distributions. As shown in Table 2, ideal particles are obtained in the combination of specific X and Y also in TX100 in the same way as when SDS is used.

As shown also in FIG. 6, it is found that as the concentration of TX100 decreases from 0.75 mM to 0.05 mM, the average particle size of the obtained particles grows from around 15 nm to around 60 nm. When the concentration of TX100 was 0 mM, the average particle diameter was less than 10 nm, and was evaluated as “poor” The distribution might exhibit multi-peakedness, or the variance might be large depending on the balance between the variation parameter X and the variation parameter Y.

TABLE 2
Variation parameter Y

| | | ECH | | | | | |
|---|---|---|---|---|---|---|---|
| | [symbol] X-100 | 2.1 ml | 2.2 ml | 2.3 ml | 2.4 ml | 2.5 ml | |
| Variation | 0 mM | Poor | Poor | Poor | Poor | Poor | Excellent → single peak |
| parameter | 0.010 mM | Good | Good | Good | Excellent | Poor | and variance of 0.25 or |
| X | (CMC × 0.043) | | | | | | less |
| | 0.060 mM | Good | Good | Excellent | Excellent | Poor | Good → two peaks or |
| | (CMC × 0.22) | | | | | | variance of 0.25 or more |
| | 0.10 mM | Good | Good | Excellent | Excellent | Good | |
| | (CMC × [symbol] ) | | | | | | |
| | [symbol] mM | Good | Excellent | Excellent | Excellent | Good | Poor → multiple peaks |
| | (CMC × [symbol] ) | | | | | | |
| | 0.75 mM | Poor | Good | Excellent | Excellent | Excellent | |
| | (CMC × [symbol] ) | | | | | | |

[symbol]

indicates data missing or illegible when filed

### Production Example 6

Particles were produced in the same way as in Example 1 except that the surfactant type was changed into hexadecyltrimethylammonium bromide (CTAB). If the variance (σ/Rₕₐᵥ) at that time was found, the variance was 0.2, and the distribution was single-peaked. The average particle diameter calculated from the local maximum of the peak was 40 nm. It can be determined that this is equivalent to “excellent” in Table 1, Table 2, and the like.

### Production Example 7

The incorporation of drugs other than α-mangostin into the particles obtained in Example 1 was attempted. At this time, the drugs were incorporated by the same method as in Production Example 3, and the loading rates thereof were determined.

“Curcumin I” is curcumin, and is a polyphenol compound contained in turmerics or the like. “Resveratrol” is resveratrol, and is one of stilbene derivative polyphenols. “THSG” is 2,3,5,4-tetrahydroxy diphenylethylene-2-O-glucoside, and is a physiologically active substance extracted from Polygonum multiflorum. “Dihydromyricetin” is dihydromyricetin, and is one of flavanols. “Astilbin” is astilbin, and is one of flavanols. “αMangostin” is α-mangostin (Production Example 3). “Dox” is doxorubicin, and is one of anti-cancer agents.

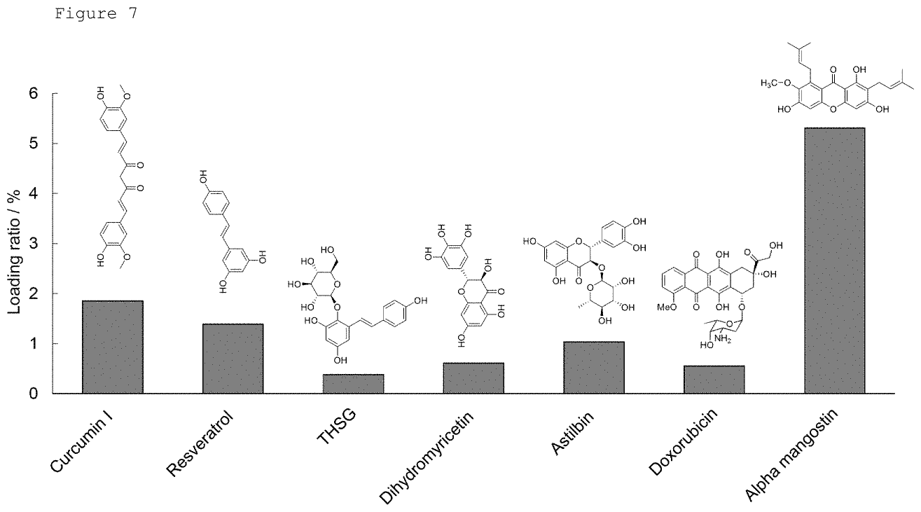

FIG. 7 shows the comparison of the loading rates of the drugs. All the drugs could be incorporated into the particles of the present invention, and the result was that the incorporation amounts thereof depended on the structures of the drug molecules.

### Production Example 8

Particles were synthesized in the same way shown in Example 1 using α-CD and γ-CD instead of β-CD, and the results obtained by evaluating the particle size were summarized in Table 3. As shown in Table 3, the average particle sizes of the obtained particles could be controlled in the range of 10 to 100 nm in the cases of both CDs.

TABLE 3
Variation parameter Y

| | | ECH: 2.4 mL | ECH: 2.4 mL | ECH: 2.4 mL | |
|---|---|---|---|---|---|
| | | SOS | Triton | CTAB | |
| | Sample | (3.0 mM) | (0.050 mM) | (0.75 mM) | |
| Variation | αCD | Excellent | Good | Good | Excellent → single peak |
| parameter | | | | | and variance of 0.25 or |
| X | | | | | less |
| | βCD | Excellent | Excellent | Excellent | Good → two peaks or |
| | | | | | variance of 0.25 or more |
| | γCD | Good | Excellent | Excellent | Poor → multiple peaks |

### [Example 2] In Vitro Experiment Using Cell Line

The mouse colorectal cancer cell CT26WT was seeded in a 96-well plate and cultured under the conditions of 37° C. and 5% CO₂ for 24 hours.

Cytotoxicity assay was performed using a high water-soluble tetrazolium salt WST-8 [a 2-(2-methoxy-4-nitrophenyl)-3-(4-nitrophenyl)-5-(2,4-disulfophenyl)-2H-tetrazolium monosodium salt], provided from a Cell Counting Kit-8 (Dojindo Molecular Technologies, Inc.).

WST-8 is reduced by dehydrogenase in cells to generate formazan (orange), and the amount of this formazan coloring matter is in direct proportion to the number of live cells.

A sample was added to cells, and the cells were then cultured for 48 hours. The amount of the formazan coloring matter was measured using a Multiskan FC spectrophotometer (Thermo Fisher Scientific K.K.), and the cell viability was determined. The concentrations of the drugs at this time were 50 μM.

The following samples was used.

Nanoparticles used in an animal experiment: average particle size of 6 nm (produced under the conditions of Production Example 2)
Drug: doxorubicin (Dox), curcumin (Cur), α-mangostin (MGS)
Final drug concentration: 50 μM
Final drug-containing nanoparticle concentration: 0.45 mg/mL (The drug concentration was 50 μM.)
Doxorubicin hydrochloride (Dox), purity: 953 or more: Tokyo Chemical Industry Co., Ltd.

### Curcumin (Cur): FUJIFILM Wako Pure Chemical Corporation

α-mangostin (MGS): Tokyo Chemical Industry Co., Ltd.

### TWEEN 80: Tokyo Chemical Industry Co., Ltd.

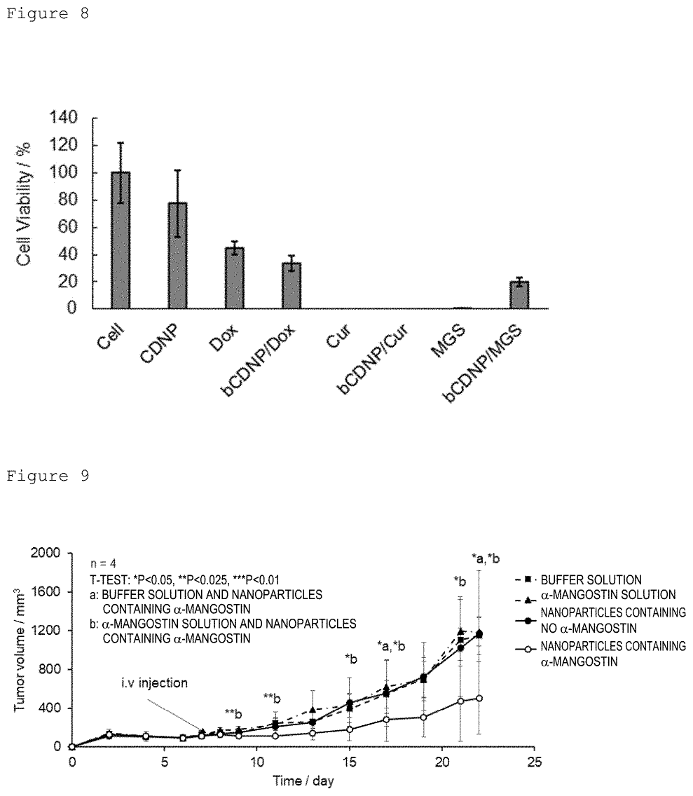

FIG. 8 shows the evaluation results. It is found that the nanoparticle (CDNP) themselves have almost no toxicity, and the toxicities of the nanoparticles containing doxorubicin (Dox), curcumin (Cur), and α-mangostin (MGS) are almost the same as the original toxicities of the drugs. This result has shown that even when the nanoparticles (CDNP) to be used in the present invention contains the drugs, the nanoparticles do not deteriorate the physiological activities thereof. That is, the expression of the physiology activities of the drugs themselves in affected parts to which the drugs are delivered can be expected.

### [Example 3] Animal Experiment Using Mice

The anti-cancer action was tested using mice. All the animal experiments shown below were conducted after the approval of the University of Kitakyushu and the animal ethics committee of the University of Kitakyushu was obtained.

The anti-tumor effect of the particles of the present invention containing α-mangostin was examined using mice (BALB/c, 7 weeks old, male) that bore the mouse colorectal cancer cell CT26. It was confirmed that the size of the tumor that the mice bore reached 100 mm³ (7 days after), and a sample was intravenously administered (i. v. injection). The doses of α-mangostin at this time were standardized to 10 mg/kg. The transition of the tumor volume was observed after the administration.

The following samples were used.

Nanoparticles: average particle size: 20 nm (produced under the conditions of X (TX100): 0.15 mM and Y (ECH): 2.1 mL of Production Example 5)
α-mangostin: Tokyo Chemical Industry Co., Ltd.
Buffer solution: phosphate buffer (pH 7.5)
nanoparticles containing no α-mangostin: only the nanoparticles (50 mg/mL)
α-mangostin solution: α-mangostin solution (1.0 mM) containing 0.4 vol % DMSO, 2 vol % ethanol, and 2 vol TWEEN 80 (surfactant)
nanoparticles containing α-mangostin: solution in which α-mangostin was contained in the nanoparticles (nanoparticle concentration: 50 mg/mL, α-mangostin concentration: 1.0 mM)
Experimental data (animal experiment using mice)

FIG. 9 shows the results obtained by observing the transition of the tumor volume after sample administration. It is found that while the effect of suppressing the cancer growth was not observed in the buffer (control), the α-mangostin solution (MGS), and the nanoparticle containing no α-mangostin (polymer), the cancer growth can be suppressed in the nanoparticles containing α-mangostin (P/MGS). When the buffer and the α-mangostin solution were compared, a significant anti-tumor effect was seen. It is believed that this is because since the particles had an average particle size of 5 nm or more using the particles of the present invention, the particles were hardly removed outside the body by renal excretion or the like, and the particles could remain in blood or the like for a long period of time. It is believed that since the particles of the present invention could avoid renal excretion, and had a size suitable for the EPR effect, the remarkable anti-cancer effect was obtained.

### INDUSTRIAL APPLICABILITY

The particles of the present invention can be used for a drug or the like containing a hydrophobic physiological active agent or the like, and are useful industrially.

1. Particles, each comprising: a polymer having a structure unit derived from a saccharide compound having hydroxyl groups and having an inclusion property and a structure unit derived from a monomer having functional groups to be reacted with hydroxyl groups, wherein an average hydrodynamic radius (Rₕₐᵥ) is 5 to 100 nm.

2. The particles according to claim 1, wherein the saccharide compound comprises a saccharide compound of any saccharide selected from the group consisting of cyclodextrin, β-1,3-glucan, and amylose.

3. The particles according to claim 1, wherein the monomer comprises a polyfunctional epoxy-based compound.

4. The particles according to claim 1, wherein a value obtained by dividing standard deviation (σ) of particle size distribution by the average hydrodynamic radius (Rₕₐᵥ) (σ/Rₕₐᵥ) is 0.25 or less when the average hydrodynamic radius (Rₕₐᵥ) is defined as a median in the particle size distribution by normal distribution.

5. A drug, comprising: a hydrophobic physiological active agent contained in the particles according to claim 1.

6. The drug according to claim 5, wherein the hydrophobic physiological active agent comprises a hydrophobic physiological active agent dispersed in one or more dispersion media selected from the group consisting of dimethyl sulfoxide, tetrahydrofuran, N,N-dimethylformamide, methanol, ethanol, propanol, and butanol.

7. An anti-cancer agent, comprising, in the drug according to claim 5, a hydrophobic physiological active agent of any selected from the group consisting of α-mangostin, curcumin, and doxorubicin and further comprising a pharmaceutically acceptable carrier.

8. The anti-cancer agent according to claim 7, wherein the cancer is a solid cancer.

9. A method for producing particles,
comprising: a step of mixing a mixed composition comprising a saccharide compound having hydroxyl groups and having an inclusion property, a monomer having functional groups to be reacted with hydroxyl groups, a surfactant, and an alkaline aqueous solution having a pH of 12 or more,
wherein the particles each comprise a polymer formed in the mixed composition and having a structure unit derived from the saccharide compound and a structure unit derived from the monomer.

10. The method for producing particles according to claim 9, wherein the surfactant comprises one or more surfactants selected from the group consisting of a quaternary ammonium salt-based surfactant, a sulfonate-based surfactant, and an oligo ethylene glycol-based surfactant.

11. The method for producing particles according to claim 10, wherein the quaternary ammonium salt-based surfactant is hexadecyltrimethylammonium bromide, the sulfonate-based surfactant is sodium dodecyl sulfate, and the oligo ethylene glycol salt-based surfactant is Triton X-100.

12. The method for producing particles according to claim 9, wherein a concentration of the surfactant in the mixed composition (mass of the surfactant/total mass of the mixed composition) is 4 times or less a critical micelle concentration of the surfactant in neutral water.

13. A method for producing a drug, wherein a first composition comprising the particles obtained by the production method according to claim 9 and a second composition comprising a hydrophobic physiological active agent are mixed to incorporate the hydrophobic physiological active agent into the particles.

14. The method for producing a drug according to claim 13, wherein the second composition comprises one or more dispersion media selected from the group consisting of dimethyl sulfoxide, tetrahydrofuran, N,N-dimethylformamide, methanol, ethanol, propanol, and butanol as a dispersion medium.
